(19) United States
(12) Patent Application Publication
(10) Pub. No.: US 2004/0210953 A1
(43) Pub. Date: Oct. 21, 2004

(54) Lysozyme transgenic ungulates

(76) Inventors: James D. Murray, Davis, CA (US); Gary B. Anderson, Davis, CA (US); Elizabeth A. Maga, Sacramento, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/753,587

(22) Filed: Jan. 7, 2004

Related U.S. Application Data
(60) Provisional application No. 60/439,304, filed on Jan. 9, 2003.
(60) Provisional application No. 60/480,355, filed on Jun. 20, 2003.

(51) Int. Cl. A01K 67/027

(52) U.S. Cl. 800/14

(57) ABSTRACT

The present invention provides transgenic ungulates that include a transgene that encodes lysozyme, and methods for producing such animals. The invention further provides methods of producing a food product, such as milk, or a milk product, using a subject transgenic ungulate, as well as food products harvested from a subject transgenic ungulate.

### CROSS-REFERENCE

This application claims the benefit of U.S. Provisional Patent Application No. 60/439,304 filed Jan. 9, 2003, and of U.S. Provisional Patent Application No. 60/480,355 filed Jun. 20, 2003, which applications are incorporated herein by reference in their entirety.

### FIELD OF THE INVENTION

The present invention is in the field of transgenic non-human animals, and milk products produced by such animals.

### BACKGROUND OF THE INVENTION

Cheese yield determines profits for the dairy industry since it represents the amount of product made from a given amount of milk. Milk composition and quality influence cheese making properties such as coagulation time (rennet clotting time), gel (curd) strength and cheese yield.

Cheese is generally made by acidifying the milk and setting the milk with a clotting agent, such as rennet, or by developing acidity to the isoelectric point of the protein. The set milk is cut and whey is separated from the resulting curd. The curd may be pressed to provide a cheese block. The whey, which contains significant amounts of whey protein, is generally further processed for protein and fat recovery. Curing typically takes place over an extended period of time (often several months or longer) under controlled conditions. Process cheese-type products can be prepared from such conventional cheeses by grinding and then heating the ground cheeses with emulsifying salt.

There have been many efforts to provide simplified processes for making cheese or cheese-type products, thereby increasing production efficiency. For example, elimination of the whey drainage step, and other modifications to the standard process have been described.

In spite of the numerous attempts and the clear advantages that a simplified process would provide, there exists a need in the art for improved methods of cheese production. The present invention addresses this need by providing lysozyme transgenic ungulates. Milk produced by a subject transgenic ungulate exhibits reduced rennet clotting time, and has enhanced bacteriostatic properties. Cheese products produced from milk of a subject transgenic ungulate also have increased gel strength. These and other advantages are discussed in detail below.

Literature

U.S. Pat. Nos. 5,891,698, 6,140,552, 6,268,487, 6,140,552, 5,741,957, 6,013,857, 6,118,045, 5,994,616, 5,907,080, 5,750,176, 5,892,070, 6,204,431; WO 97/05771; WO 01/00855; Gutierrez-Adan et al. (1999) J. Dairy Res. 66:289-294; Maga et al. (1998) J. Food Prot. 61:52-56; Maga et al. (1995) J. Dairy Sci. 78:2645-2652; Maga and Murray (1995) Bio/Technology 13:1452-1457; Maga et al. (1994) Transgenic Res. 3:36-42.

### SUMMARY OF THE INVENTION

The present invention provides transgenic ungulates that include a transgene that encodes lysozyme, and methods for producing such animals. The invention further provides methods of producing a food product, such as milk, or a milk product, using a subject transgenic ungulate, as well as food products harvested from a subject transgenic ungulate.

### FEATURES OF THE INVENTION

The present invention features a transgenic ungulate that includes a transgene encoding lysozyme. In some embodiments, the ungulate is female, the lysozyme-encoding transgene is expressed in mammary gland cells of the ungulate, and the milk produced by the transgenic ungulate has a level of lysozyme that is at least about 10% higher than the level of lysozyme in milk of a non-transgenic ungulate of the same species. In some of these embodiments, the transgene comprises a coding sequence for lysozyme operably linked to a mammary specific promoter. In other embodiments, the ungulate is male. In some embodiments, the ungulate is a goat.

In some embodiments, the transgene is chromosomally integrated. In some embodiments, the ungulate is heterozygous for the transgene. In other embodiments, the ungulate is homozygous for the transgene. In some embodiments, the transgene encodes human lysozyme.

The present invention further features an isolated fertilized egg, where the egg is isolated from a transgenic ungulate comprising a transgene encoding lysozyme. The present invention further features a composition comprising an isolated fertilized egg of the present invention; and a cryoprotective agent.

The present invention further features a method of producing a food product, the method involving harvesting a food product from a transgenic ungulate of the present invention.

The present invention further features a method of producing a food product, the method involving processing a food product harvested from a transgenic ungulate of the present invention.

The present invention further features a method of producing cheese, the method involving: adding rennet to milk harvested from a transgenic ungulate of the present invention; allowing curd formation to occur; and producing cheese from the curds.

The present invention further features a milk product produced by a transgenic ungulate of the present invention, where the milk product has a level of lysozyme that is at least about 10% higher than the level of lysozyme in milk of a non-transgenic ungulate of the same species. In some embodiments, the milk product has reduced rennet clotting time compared to a milk product of a non-transgenic animal of the same species. In some embodiments, the milk product has enhanced bacteriostatic properties compared to a milk product of a non-transgenic animal of the same species. In some embodiments, the milk product has increased shelf life compared to the shelf life of the same milk product made from milk of a non-transgenic animal of the same species.

The present invention further features a processed milk produced from milk of a transgenic ungulate of the present invention. In some embodiments, the processed milk is cheese. In some of these embodiments, the cheese has increased gel strength compared to cheese made from milk of a non-transgenic ungulate of the same species.

### Definitions

The term “transgene” is used herein to describe genetic material which has been or is about to be artificially inserted into the genome of a non-human animal, and particularly into a cell of a living non-human mammal.

The term “transformation” refers to a permanent or transient genetic change induced in a cell following the incorporation of new DNA (i.e. DNA exogenous to the cell). Where the cell is a mammalian cell, a permanent genetic change is generally achieved by introduction of the DNA into the genome of the cell.

The term “ES cell” as used herein refers to pluripotent embryonic stem cells and to such pluripotent cells in the very early stages of embryonic development, including but not limited to cells in the blastocyst stage of development.

The term “construct” refers to a recombinant nucleic acid, generally recombinant DNA, that has been generated for the purpose of the expression of a specific nucleotide sequence(s), or is to be used in the construction of other recombinant nucleotide sequences.

The term “operably linked” refers to a functional connection between a DNA sequence and a regulatory sequence(s), e.g., a DNA sequence and a regulatory sequence(s) are connected in such a way as to permit gene expression when the appropriate molecules (e.g., transcriptional activator proteins) are bound to the regulatory sequence(s).

The term “cDNA” refers to all nucleic acids that share the arrangement of sequence elements found in native mature mRNA species, where sequence elements are exons and 3′ and 5′ non-coding regions. Normally mRNA species have contiguous exons, with the intervening introns removed by nuclear RNA splicing, to create a continuous open reading frame encoding the protein.

The term “genomic sequence” refers to a sequence having non-contiguous open reading frames, where introns interrupt the protein coding regions. It may further include the 3′ and 5′ untranslated regions found in the mature mRNA. It may further include specific transcriptional and translational regulatory sequences, such as promoters, enhancers, etc., including about 1 kb, but possibly more, of flanking genomic DNA at either the 5′ or 3′ end of the transcribed region. The genomic DNA may be isolated as a fragment of 100 kbp or smaller; and substantially free of flanking chromosomal sequence.

### DETAILED DESCRIPTION OF THE INVENTION

The present invention provides transgenic ungulates that include a transgene that encodes lysozyme, and methods for producing such animals. The subject transgenic animals are useful for producing milk. The milk produced by a subject transgenic animal has a higher level of lysozyme than milk produced by a non-transgenic animal of the same species. The elevated level of lysozyme confers various advantages on the milk. For example, the milk has a reduced rennet clotting time, and is therefore advantageous for production of cheese, as cheese yield is increased. Furthermore, the cheese produced by such milk has increased gel (curd) strength. The lysozyme is also bacteriostatic toward a variety of undesirable bacteria, and therefore the presence of increased levels of lysozyme in the milk increases food safety. Furthermore, the bacteriostatic properties of the lysozyme reduce the incidence of mammary gland infections (mastitis) in the milk-producing transgenic ungulate.

The subject invention further provides methods of producing a milk from a subject transgenic ungulate, by harvesting the milk from the subject transgenic ungulate. The subject invention also provides methods of making food products from the milk of a subject transgenic ungulate. The subject invention further provides milk harvested from a subject transgenic ungulate, and food products made with such milk.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a transgene” includes a plurality of such transgenes and reference to “the milk product” includes reference to one or more milk products and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Transgenic Non-Human Animals and Methods for Their Production

The present invention provides transgenic ungulates that include a lysozyme transgene. A lysozyme transgene includes a nucleotide sequence that encodes lysozyme. The lysozyme coding sequence is any coding sequence that, when transcribed and translated, provides for production of enzymatically active lysozyme. The lysozyme coding sequence is operably linked to one or more control elements that provide at least for transcription of the coding sequence. In many embodiments, e.g., where the transgenic ungulate is a female, the lysozyme coding sequence is operably linked to a mammary gland-specific promoter such that the encoded lysozyme is produced in the mammary gland. In some embodiments, male lysozyme transgenic ungulates also include a lysozyme transgene in which the lysozyme coding sequence is operably linked to a mammary gland-specific promoter. Suitable coding sequences and promoters are described in more detail below.

The transgenic ungulates of the present invention are milk-producing agricultural ungulates, including, but not limited to, goats, sheep, and cows. The present invention provides both female and male lysozyme transgenic animals. A subject transgenic animal is heterozygous or homozygous for the lysozyme transgene. Males are useful for breeding with female ungulates of the same species to produce offspring that are homozygous or heterozygous for the lysozyme transgene. Females are useful both for production of milk with increased levels of lysozyme, and for production of transgenic offspring.

A subject female transgenic ungulate produces milk that has a level of enzymatically active lysozyme that is at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 4-fold, at least about 5-fold, at least about 10-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1000-fold, at least about 1200-fold, at least about 1500-fold, at least about 1700-fold, at least about 2000-fold, at least about 2500-fold, or at least about 3000-fold, or more, higher than the level of enzymatically active lysozyme in the milk of a female non-transgenic animal of the same species. For example, the level of enzymatically active lysozyme in the milk of a subject transgenic ungulate is from about 0.10 mg/ml to about 2.0 mg/ml, from about 0.2 mg/ml to about 1.0 mg/ml, or from about 0.4 mg/ml to about 0.8 mg/ml. In some embodiments, the level of enzymatically active lysozyme in the milk of a subject transgenic ungulate is greater than 2.0 mg/ml, e.g., from about 2.0 mg/ml to about 7.5 mg/ml, e.g., from about 2.0 mg/ml to about 2.5 mg/ml, from about 2.5 mg/ml to about 3.0 mg/ml, from about 3.0 mg/ml to about 3.5 mg/ml, from about 3.5 mg/ml to about 4.0 mg/ml, from about 4.0 mg/ml to about 5.0 mg/ml, from about 5.0 mg/ml to about 6.0 mg/ml, from about 6.0 mg/ml to about 7.0 mg/ml, or from about 7.0 mg/ml to about 7.5 mg/ml.

Methods of measuring the level of lysozyme polypeptide, and methods of measuring the level of enzymatically active lysozyme, in milk are known in the art, and any known method can be used. Suitable methods for measuring the level of lysozyme polypeptide in milk include, but are not limited to, Western blotting (e.g., applying a sample to a polyacrylamide gel, electrophoresing the sample, transferring proteins in the gel to a membrane, and detecting lysozyme on the membrane using an antibody specific for lysozyme), followed by densitometric scanning; an enzyme linked immunosorbent assay (ELISA) using antibody specific for lysozyme; and the like. Suitable methods for measuring enzymatic activity of lysozyme include, but are not limited to, an assay for clearing of bacteria incorporated into a gel; bacterial clearing in solution (using a turbidometric assay); and the like. In many assays, lysozyme activity is measured by clearing of Micrococcus lysodeikticus.

A subject transgenic ungulate produces milk that is bacteriostatic, particularly toward Gram-positive bacteria. Milk from a subject transgenic ungulate is at least about 5%, at least about 10%, at least about 15%, at least about 20%, at least about 25%, at least about 30%, at least about 35%, at least about 40%, at least about 45%, or at least about 50%, at least about 60%, at least about 70%, at least about 80%, at least about 90%, at least about 2-fold, at least about 4-fold, at least about 5-fold, at least about 10-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1000-fold, at least about 1200-fold, at least about 1500-fold, at least about 1700-fold, at least about 2000-fold, at least about 2500-fold, or at least about 3000-fold, or more, bacteriostatic than milk from a non-transgenic ungulate of the same species. In particular, the milk of a subject ungulate exhibits bacteriostatic activity toward mastitis-causing bacteria, and toward bacteria that cause food spoilage. Such bacteria include, but are not limited to, Escherichia coli; various staphylococcal species, including, e.g., Staphylococcus areus; Pseudomonas species, including, e.g., Pseudomonas fragi, P. fluorescens, P. aeruginosa; streptococcal species, including e.g., Streptococcus cremoris, S. agalactiae, S. uberis; Lactococcus species, including, e.g., L. lactis; Lactobacillus species; and the like.

The level of bacteriostatic activity of a sample of milk is readily determined using well-known assays. For example, a bacterial suspension is added to a milk sample, and the milk sample is incubated at 37° C. for various periods of time, after which times dilutions of the cultures are plated onto agar containing bacterial growth medium, and the number of bacteria per culture determined. The level of bacteriostatic activity of the milk sample is inversely related to the number of bacteria.

The milk, or a product made from milk of a subject transgenic ungulate has increased shelf life compared to the shelf life of the same milk product made from milk of a non-transgenic animal of the same species. For example, the shelf life of milk or a product made from the milk of a subject transgenic ungulate has a shelf life that is at least about 10%, at least about 15%, at least about 20%, at least about 25%, at least about 30%, at least about 35%, at least about 40%, at least about 50%, at least about 60%, at least about 70%, at least about 80%, at least about 90%, at least about 2-fold, at least about 3-fold, at least about 4-fold, or at least about 5-fold or more greater than the shelf life of the milk or the same milk product made from milk of a non-transgenic animal of the same species.

The milk of a subject transgenic ungulate has reduced rennet clotting time, compared to the milk of a non-transgenic ungulate of the same species. The rennet clotting time is the time required for curd formation to occur. A reduction in rennet clotting time provides for enhanced cheese production. The rennet clotting time of milk of a subject transgenic ungulate is reduced by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%, or more, compared to the rennet clotting time of milk of a non-transgenic ungulate of the same species.

Rennet clotting time is determined using any known method, including the method described in the Example. Methods for determining rennet clotting time are known in the art. See, e.g., Maga et al. (1995) J. Dairy Sci. 78:2645-2652. Rennet clotting times of two or more milk samples are compared under the same temperature conditions, and curd formation is measured at a given time after addition of rennet.

Cheese produced from the milk of a subject transgenic ungulate has increased gel strength compared to cheese made by the same process from milk of a non-transgenic ungulate of the same species. The gel strength of cheese produced from the milk of a subject transgenic ungulate is at least about 5%, at least about 10%, at least about 15%, at least about 20%, at least about 25%, at least about 30%, at least about 35%, at least about 40%, at least about 45%, at least about 50%, or more, greater than the gel strength of cheese made by the same process from milk of a non-transgenic ungulate of the same species. Increased gel strength is measured using any known method, e.g., the method described in the Example.

Methods of Making a Subject Transgenic Animal

The invention provides methods of generating a subject transgenic animal. The method generally involves introducing a lysozyme transgene into a pluripotent or totipotent cell such that the transgene is integrated into the genome of the cell, and transferring the cell into an oviduct of a synchronized recipient female of the same species as the cell.

A transgene that includes a coding region for lysozyme is used to transform a cell, meaning that a permanent or transient genetic change, generally a permanent genetic change, is induced in a cell following incorporation of the exogenous DNA of the transgene. A permanent genetic change is generally achieved by introduction of the DNA into the genome of the cell. Vectors for stable integration include plasmids, retroviruses and other animal viruses, BACs, HACs, YACs, and the like.

Transgenic animals of the invention comprise an exogenous nucleic acid sequence present as an extrachromosomal element or stably integrated in all or a portion of its cells, especially in germ cells. Unless otherwise indicated, it will be assumed that a transgenic animal comprises stable changes to the germline sequence. A subject transgenic animal may be heterozygous or homozygous for the transgene. During the initial construction of the animal, “chimeras” or “chimeric animals” are generated in some methods (e.g., where ES cells are used), in which only a subset of cells have the altered genome. Chimeras are primarily used for breeding purposes in order to generate the desired transgenic animal. Animals having a heterozygous alteration are generated by breeding of chimeras. Male and female heterozygotes are typically bred to generate homozygous animals.

In some embodiments, the lysozyme transgene that is introduced into a cell includes an exogenous lysozyme coding sequence. The exogenous gene is in some embodiments from a different species than the animal host (e.g., is a heterologous lysozyme gene). The exogenous gene may or may not be altered in its coding sequence. Non-coding sequences, such as control elements, may or may not be present. Control elements, if present in the transgene, include homologous (e.g., normally associated with the coding sequence) or heterologous (e.g., not normally associated with the coding region, e.g., from another species). The introduced gene may be a wild-type gene, naturally occurring polymorphism, or a genetically manipulated sequence, for example having deletions, substitutions or insertions in the coding or non-coding regions. The lysozyme coding region may be operably linked to a promoter, which may be constitutive or inducible, and other regulatory sequences required for expression in the host animal. Alternatively, the lysozyme coding region may not be operably linked to a control element(s) in the transgene, but instead becomes operably linked to control element(s) when it becomes integrated into the genome. By “operably linked” is meant that a DNA sequence and a regulatory sequence(s) are connected in such a way as to permit gene expression when the appropriate molecules, e.g. transcriptional activator proteins, are bound to the regulatory sequence(s).

In other embodiments, the lysozyme transgene that is introduced into a cell includes an endogenous lysozyme coding sequence. In these embodiments, the coding sequence may or may not be operably linked to control element(s). The lysozyme coding region may be operably linked to a promoter, which may be constitutive or inducible, and other regulatory sequences required for expression in the host animal. Alternatively, the lysozyme coding region may not be operably linked to a control element(s) in the transgene, but instead becomes operably linked to control element(s) when it becomes integrated into the genome.

In some embodiments, a subject transgenic animal is produced by introducing into a single cell embryo a polynucleotide that comprises a nucleotide sequence that encodes lysozyme, or fragments or variants thereof, in a manner such that the polynucleotide is stably integrated into the DNA of germ line cells of the mature animal, and is inherited in normal Mendelian fashion. In accordance with the invention, a polynucleotide can be introduced into an embryo by a variety of means to produce transgenic animals. For instance, totipotent or pluripotent stem cells or somatic cells can be transformed by microinjection, calcium phosphate mediated precipitation, liposome fusion, retroviral infection or by other means. The transformed cells can then be introduced into embryos and incorporated therein to form transgenic animals.

In many embodiments, a polynucleotide is injected into an embryo, e.g., at the single-cell stage, forming a genetically modified embryo, and the genetically modified embryo is allowed to develop into a mature transgenic animal.

In some embodiments, the transgene is introduced into a somatic cell, where the transgene is integrated into the genome, forming a genetically modified somatic cell, and the nucleus of the genetically modified somatic cell is transferred into a single-cell embryo, forming a genetically modified embryo. The genetically modified single-cell embryo is then transferred into an oviduct of a recipient female, and the embryo allowed to develop into a mature transgenic animal.

Any method of making transgenic animals can be used as described, for example, in Transgenic Animal Generation and Use L. M. Houdebine, Harwood Academic Press, 1997; Transgenesis Techniques: Principles and Protocols D. Murphy and D. A. Carter, ed. (June 1993) Humana Press; Transgenic Animal Technology: A Laboratory Handbook C. A. Pinkert, ed. (January 1994) Academic Press; Transgenic Animals F. Grosveld and G Kollias, eds. (July 1992) Academic Press; and Embryonal Stem Cells: Introducing Planned Changes into the Animal Germline M. L. Hooper (January 1993) Gordon & Breach Science Pub; U.S. Pat. No. 6,344,596; U.S. Pat. No. 6,271,436; U.S. Pat. No. 6,218,596; and U.S. Pat. No. 6,204,431; Maga and Murray (1995) Bio/Technol. 13:1452-1457; Ebert et al. (1991) Bio/Technol. 9:835-838; Velander et al. (1992) Proc. Natl. Acad. Sci. USA 89:12003-12007; Wright et al. (1991) Bio/Technol. 9:830-834.

Transgenic animals also can be generated using methods of nuclear transfer or cloning using embryonic or adult cell lines as described for example in Campbell et al. (1996) Nature 380: 64-66; and Wilmut et al. (1997) Nature 385: 810-813. Cytoplasmic injection of DNA can be used, as described in U.S. Pat. No. 5,523,222. Subject transgenic animals can be obtained by introducing a chimeric construct comprising lysozyme-encoding sequences.

Transgenic animals also include somatic transgenic animals, e.g., transgenic animals that include a transgene in somatic cells (and not in germ line cells). For example, the mammary gland cells of an animal are transformed with a lysozyme transgene, and the transgene is expressed in mammary cells of the animal. Methods of somatic cell transformation are described in the art. See, e.g., Furth et al. (1995) Mol. Biotechnol. 4:121-127.

Methods for making transgenic goats are known in the art. See, e.g., Zou et al. (2002) Mol. Reprod. Dev. 61:164-172; Baldassare et al. (2002) Theriogenol. 57:275-284; and Ko et al. (2000) Transgenic Res. 9:215-222. Methods for making lysozyme transgenic goats are also described in the Examples. Methods for making transgenic cows are known in the art, and are described in, e.g., van Berkel et al. (2002) Nat. Biotechnol. 20:484-487.

Expression Vectors and Transgenes

A subject transgenic animal is typically generated by a method involving introducing into a cell a construct comprising a nucleotide sequence encoding lysozyme. A “lysozyme transgene” includes, at a minimum, a coding region for lysozyme. In some embodiments, the nucleotide sequence encoding lysozyme is operably linked to a promoter and, optionally, additional control elements, that provide for tissue-specific expression of the transgene in the animal. In other embodiments, the nucleotide sequence encoding lysozyme is not operably linked to any control elements. Instead, the lysozyme transgene includes, on the 5′ and 3′ ends of the coding region, sequences that provide for homologous recombination with an endogenous gene.

Any known coding sequence for lysozyme can be used to make a subject transgenic animal, including a lysozyme coding sequence from rat, mouse, human, cow, goat, sheep, etc. The coding sequence can be a cDNA sequence, or a genomic sequence. The coding sequence for the lysozyme may be, but need not be, from the same species as the transgenic animal. In many embodiments, the lysozyme transgene includes a coding region for human lysozyme.

The nucleotide sequences of mRNAs encoding lysozyme from a variety of animal species are known. Exemplary sequences are found under the following GenBank Accession numbers: mouse lysozyme mRNA, NM_{—}017372; rat lysozyme mRNA, NM_{—}012594 and L12458; human lysozyme mRNA, NM_{—}000239.

In addition, sequences that vary from a known coding sequence for lysozyme can be used, as long as the encoded lysozyme has substantially the same enzymatic activity. For example, the encoded lysozyme can include one or more conservative amino acid substitutions compared to the amino acid sequence of a known lysozyme. Examples of conservative amino acid substitutions are Phe/Tyr; Ala/Val; Leu/Ile; Arg/His; Ser/Thr; etc. The encoded lysozyme can also include insertions or deletions (including truncations) of one or more amino acid residues, compared to the amino acid sequence of a known lysozyme. Further, the encoded lysozyme can include one or more naturally occurring polymorphisms.

A suitable nucleotide sequence encoding a lysozyme generally has at least about 75%, at least about 80%, at least about 85%, at least about 90%, at least about 95%, or at least about 98%, or higher, nucleotide sequence identity with a known coding sequence for lysozyme. Sequence similarity is calculated based on a reference sequence, which may be a subset of a larger sequence, such as a conserved motif, coding region, flanking region, etc. A reference sequence will usually be at least about 18 nt long, more usually at least about 30 nt long, and may extend to the complete sequence that is being compared. Algorithms for sequence analysis are known in the art, such as BLAST, described in Altschul et al. (1990), J. Mol. Biol. 215:403-10 (using default settings).

Also suitable for use are lysozyme coding sequences that hybridize under stringent hybridization conditions to a known lysozyme coding sequence. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 1×SSC (150 mM NaCl, 15 mM sodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C. For example, high stringency conditions include aqueous hybridization (e.g., free of formamide) in 6×SSC (where 20×SSC contains 3.0 M NaCl and 0.3 M sodium citrate), 1% sodium dodecyl sulfate (SDS) at 65° C. for about 8 hours (or more), followed by one or more washes in 0.2×SSC, 0.1% SDS at 65° C. For example, moderate stringency conditions include aqueous hybridization (e.g., free of formamide) in 6×SSC, 1% SDS at 65° C. for about 8 hours (or more), followed by one or more washes in 2×SSC, 0.1% SDS at room temperature.

As noted above, in some embodiments, a lysozyme transgene includes a coding sequence for lysozyme operably linked to one or more control sequences, e.g., promoters, 3′ transcriptional control sequences, translational control elements, etc. In some embodiments, a lysozyme transgene will include a control element that provides for increased mRNA stability.

In many embodiments, a lysozyme transgene includes a coding region for lysozyme operably linked to one or more tissue-specific control elements, e.g., a tissue-specific promoter, and optionally additional tissue-specific control elements (e.g., a 3′ untranslated region, an enhancer, and the like). The tissue-specific control element(s) can be heterologous, e.g., not normally operably linked to a lysozyme coding sequence in nature, or homologous, e.g., normally operably linked to a lysozyme coding sequence in nature. Tissue-specific control elements provide for expression of the lysozyme transgene preferentially in a given tissue, e.g., such control elements are more active (e.g., 2-fold, 5-fold, 10-fold, 20-fold, or 50-fold more active, or greater than 50-fold more active) in a given tissue than in other tissues under normal physiological conditions. A wide variety of tissue-specific promoters are known in the art.

Promoters useful for production of lysozyme in the milk of a subject transgenic animal are active in mammary tissue, e.g., the promoters are more active in mammary tissue than in other tissues under physiological conditions in which milk is synthesized. Suitable promoters provide for both specific and efficient transcription in mammary tissue. Mammary gland-specific promoters are strong promoters in mammary tissue that can support the synthesis of large amounts of protein for secretion into milk. Mammary gland-specific promoters include, but are not limited to, a whey acidic protein (WAP) promoter; αS1 casein, αS2 casein, β casein, and kappa casein promoters; an α-lactalbumin promoter; and a β-lactoglobulin (“BLG”) promoter. The sequences of a number of mammary gland-specific promoters have been isolated and their nucleotide sequences have been published. See, for example, Clark et al. (1987) TIBTECH 5:20; and Henninghausen (1990) Protein Expression and Purification 41:3.

Where the control element operably linked to the lysozyme coding region in the transgene is a lysozyme control element, the lysozyme control element may be altered to provide for increased transcription, increased mRNA stability, and the like, e.g., using random or site-specific mutagenesis techniques. Methods for random and site-specific mutagenesis are well known in the art. Whether a given mutation of a control element increases the level of lysozyme mRNA is readily determined using well-known methods. For example, an expression vector that includes a lysozyme promoter operably linked to a reporter gene, e.g., a nucleotide sequence encoding a detectable protein, such as a luciferase-encoding sequence, is introduced into a eukaryotic cell, and the promoter activity is determined by measuring the level of luciferase produced in the cell.

In some embodiments, a lysozyme transgene is not operably linked to a control element. Instead, the transgene includes sequences that provide for homologous recombination with an endogenous lysozyme gene, such that the lysozyme coding sequence replaces all or part of the endogenous lysozyme coding sequence, and the integrated lysozyme coding region is under transcriptional control of endogenous control element(s).

A lysozyme transgene is generally provided as part of a vector (e.g., a lysozyme construct), a wide variety of which are known in the art and need not be elaborated upon herein. Vectors include, but are not limited to, plasmids; cosmids; viral vectors; artificial chromosomes (HACs, YACs, BACs, etc.); mini-chromosomes; and the like. Vectors are amply described in numerous publications well known to those in the art, including, e.g., Short Protocols in Molecular Biology, (1999) F. Ausubel, et al., eds., Wiley & Sons. Vectors may provide for expression of the subject nucleic acids, may provide for propagating the subject nucleic acids, or both.

For expression, e.g., where the transgene includes a promoter, an expression cassette may be employed. The expression vector will provide a transcriptional and translational initiation region, which may be inducible or constitutive, where the coding region is operably linked under the transcriptional control of the transcriptional initiation region, and a transcriptional and translational termination region. These control regions may be native to a gene encoding the subject peptides, or may be derived from exogenous sources.

Where the transgene includes a promoter, an expression vector will generally have convenient restriction sites located near the promoter sequence to provide for the insertion of nucleic acid sequences encoding lysozyme. A selectable marker operative in the expression host may be present. Expression vectors may be used for the production of fusion proteins, where the exogenous fusion peptide provides additional functionality, i.e. increased protein synthesis, stability, reactivity with defined antisera, an enzyme marker, e.g. β-galactosidase, etc.

Expression cassettes may be prepared comprising a transcription initiation region, the gene or fragment thereof, and a transcriptional termination region.

Fertilized Eggs

The present invention further provides isolated fertilized eggs derived from a subject female transgenic ungulate. The term “fertilized egg” includes a fertilized egg at any stage after fertilization and prior to implantation, e.g., from a fertilized one-cell egg to the blastocyst stage.

A subject isolated fertilized egg is useful for generating a subject transgenic ungulate. Thus, a subject isolated fertilized egg is capable of developing into a lysozyme transgenic animal when placed in the uterus of a recipient animal of the same species, e.g., a pseudopregnant recipient female animal of the same species. The term “isolated,” in the context of a fertilized egg, refers to a fertilized egg that has been removed from a subject transgenic ungulate.

A subject isolated fertilized egg may be obtained by flushing of the uterus after fertilization and retrieval of fertilized eggs. A subject isolated fertilized egg may be stored for an extended period of time, or used immediately.

Where the subject isolated fertilized egg is stored, the fertilized egg is stored in a frozen, refrigerated, or vitrified state. Where the isolated fertilized egg is frozen, a suitable cryoprotectant compound is typically added to the fertilized egg or the medium containing the fertilized egg. Suitable cryoprotective compounds include permeating and nonpermeating compounds. Most commonly, dimethyl sulfoxide (DMSO), glycerol, propylene glycol, ethylene glycol, or the like are used as permeating cryoprotective agents. Other permeating agents include propanediol, dimethylformamide and acetamide. Nonpermeating agents include polyvinyl alcohol, polyvinyl pyrrolidine, anti-freeze fish or plant proteins, carboxymethylcellulose, serum albumin, hydroxyethyl starch, Ficoll, dextran, gelatin, albumin, egg yolk, milk products, lipid vesicles, or lecithin. Adjunct compounds that may be added include sugar alcohols, simple sugars (e.g., sucrose, raffinose, trehalose, galactose, and lactose), glycosaminoglycans (e.g., heparin, chrondroitin sulfate), butylated hydroxy toluene, detergents, free-radical scavengers, and anti-oxidants (e.g., vitamin E, taurine), and amino acids (e.g., glycine, glutamic acid).

Following suspension of the cells in the cryoprotective medium (e.g., for storage), the container is sealed and subsequently either refrigerated or frozen. Briefly, for refrigeration, the sample is placed in a refrigerator in a container filled with water for one hour or until the temperature reaches 4° C. If the sample is to be frozen, the cold sample is aliquoted into cryovials or straws and placed in the vapor phase of liquid nitrogen for one to two hours, and then plunged into the liquid phase of liquid nitrogen for long-term storage or frozen in a programmable computerized freezer. Frozen samples are thawed by warming in a 37° C. water bath and are directly used, or washed, and then used. Other cooling and freezing protocols may be used. Vitrification involves dehydration of the fertilized egg using sugars, Ficoll, or the like. The oocyte or embryo is then added to a cryoprotectant and rapidly moved into liquid nitrogen. Refrigeration is generally an appropriate means for short-term storage, while freezing or vitrification are generally appropriate means for long or short-term storage.

Utility

The subject transgenic animals find use in a variety of applications, including, but not limited to, milk production, processed milk product production, research, and the like. For example, the subject animals find use in producing milk that has decreased rennet clotting time and that results in increased efficiency of cheese production. The subject animals find use in research, to analyze the effects of lysozyme levels on production of milk and milk-based products.

Food Applications

The present invention provides methods for producing milk from a subject transgenic animal, as well as methods for producing milk-based food products from milk harvested from a subject transgenic animal. The methods generally involve harvesting milk from a subject transgenic animal. Where the food product requires further processing, the methods involve harvesting milk from a subject transgenic animal; and processing the food product.

The invention further provides milk produced by a subject transgenic ungulate, as well as food products made from milk harvested from a subject transgenic ungulate. A subject food product made from milk harvested from a transgenic ungulate includes a food product that contains a milk of a subject transgenic ungulate, or a product produced from the milk of a subject transgenic animal. Food products include any preparation for human consumption including for enteral or parenteral consumption, which when taken into the body (a) serve to nourish or build up tissues or supply energy and/or (b) maintain, restore or support adequate nutritional status or metabolic function. Food products of the invention are suitable for human consumption.

A subject food product includes milk, and any food products made from or containing milk, including, but not limited to, cheese, yogurt, butter, ice cream, and other frozen desserts, whipped toppings, cream, custard, pudding, nutritional drinks, infant formula, and chocolate.

Milk produced by a subject transgenic ungulate has a level of enzymatically active lysozyme that is at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 4-fold, at least about 5-fold, at least about 10-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1000-fold, at least about 1200-fold, at least about 1500-fold, at least about 1700-fold, at least about 2000-fold, at least about 2500-fold, or at least about 3000-fold, or more, higher than the level of enzymatically active lysozyme in the milk of a female non-transgenic animal of the same species. For example, the level of enzymatically active lysozyme in the milk of a subject transgenic ungulate is from about 0.10 mg/ml to about 2.0 mg/ml, from about 0.2 mg/ml to about 1.0 mg/ml, or from about 0.4 mg/ml to about 0.8 mg/ml. In some embodiments, the level of enzymatically active lysozyme in the milk of a subject transgenic ungulate is greater than 2.0 mg/ml, e.g., from about 2.0 mg/ml to about 7.5 mg/ml, e.g., from about 2.0 mg/ml to about 2.5 mg/ml, from about 2.5 mg/ml to about 3.0 mg/ml, from about 3.0 mg/ml to about 3.5 mg/ml, from about 3.5 mg/ml to about 4.0 mg/ml, from about 4.0 mg/ml to about 5.0 mg/ml, from about 5.0 mg/ml to about 6.0 mg/ml, from about 6.0 mg/ml to about 7.0 mg/ml, or from about 7.0 mg/ml to about 7.5 mg/ml.

Milk produced by a subject transgenic ungulate is at least about 5%, at least about 10%, at least about 15%, at least about 20%, at least about 25%, at least about 30%, at least about 35%, at least about 40%, at least about 45%, or at least about 50%, at least about 60%, at least about 70%, at least about 80%, at least about 90%, at least about 2-fold, at least about 4-fold, at least about 5-fold, at least about 10-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1000-fold, at least about 1200-fold, at least about 1500-fold, at least about 1700-fold, at least about 2000-fold, at least about 2500-fold, or at least about 3000-fold, or more, bacteriostatic than milk from a non-transgenic ungulate of the same species. In particular, the milk of a subject ungulate exhibits bacteriostatic activity toward mastitis-causing bacteria, and toward bacteria that cause food spoilage. Such bacteria include, but are not limited to, Escherichia coli; various staphylococcal species, including, e.g., Staphylococcus areus; Pseudomonas species, including, e.g., Pseudomonas fragi, P. fluorescens, P. aeruginosa; streptococcal species, including e.g., Streptococcus cremoris, S. agalactiae, S. uberis; Lactococcus species, including, e.g., L. lactis; Lactobacillus species; and the like.

The milk, or a product made from milk of a subject transgenic ungulate has increased shelf life compared to the shelf life of the same milk product made from milk of a non-transgenic animal of the same species. For example, the shelf life of milk or a product made from the milk of a subject transgenic ungulate has a shelf life that is at least about 10%, at least about 15%, at least about 20%, at least about 25%, at least about 30%, at least about 35%, at least about 40%, at least about 50%, at least about 60%, at least about 70%, at least about 80%, at least about 90%, at least about 2-fold, at least about 3-fold, at least about 4-fold, or at least about 5-fold or more greater than the shelf life of the milk or the same milk product made from milk of a non-transgenic animal of the same species.

The milk of a subject transgenic ungulate has reduced rennet clotting time, compared to the milk of a non-transgenic ungulate of the same species. The rennet clotting time is the time required for curd formation to occur. A reduction in rennet clotting time provides for enhanced cheese production. The rennet clotting time of milk of a subject transgenic ungulate is reduced by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%, or more, compared to the rennet clotting time of milk of a non-transgenic ungulate of the same species.

Cheese produced from the milk of a subject transgenic ungulate has increased gel strength compared to cheese made by the same process from milk of a non-transgenic ungulate of the same species. The gel strength of cheese produced from the milk of a subject transgenic ungulate is at least about 5%, at least about 10%, at least about 15%, at least about 20%, at least about 25%, at least about 30%, at least about 35%, at least about 40%, at least about 45%, at least about 50%, or more, greater than the gel strength of cheese made by the same process from milk of a non-transgenic ungulate of the same species.

Methods of Making Cheese

Methods of making cheese are known in the art, and a subject food product harvested from a subject ungulate can be used in the production of cheese using any known method. Rennet is added to milk from a subject transgenic ungulate, using standard industry methods. One or more additional curd formation-inducing agents can be added. Curd formation inducing agents include polycations other than lysozyme, including, but not limited to, salmine and calcium.

In general, a subject method of making cheese involves adding rennet or another curd formation inducing agent to milk from a subject transgenic ungulate; allowing curd formation to occur; and producing cheese from the curds. In some embodiments, the method involves adjusting the pH of the milk harvested from a subject transgenic animal; adding one or more curd formation inducing agents; allowing curd formation to occur; separating curd from whey; and forming the curd into a cheese product. The process may further include a curing step.

Typically, rennet is added in an amount of from about 1:1000 to about 1:15000 rennet to milk, e.g., from about 1:1000 to about 1:2000, from about 1:2000 to about 1:4000, from about 1:4000 to about 1:6000, from about 1:6000 to about 1:8000, from about 1:8000 to about 1:10,000, or from about 1:10,000 to about 1:15,000, although ratios of rennet:milk that are lower or higher can also be used. Curd formation is allowed to proceed at a temperature of from about 32° C. to about 35° C., although higher or lower temperatures can also be used, e.g. curd formation can be allowed to proceed at a temperature of from about 25° C. to about 32° C., or from about 35° C. to about 42° C. Curd formation is allowed to proceed for a period of time of from about 5 minutes to about 30 minutes, although shorter or longer curd formation times can be used, e.g., curd formation can be allowed to proceed for a period of from about 1 minute to about 60 minutes.

In some embodiments, depending on the type of cheese being produced, the pH is adjusted to an acidic pH (e.g., lower than about 6.0, e.g., to a pH of from about 2.0 to about 3.0, from about 3.0 to about 4.0, from about 4.0 to about 5.0, or from about 5.0 to about 6.0). Adjustment of pH is made by introducing an acidic agent, such as HCl, citric acid, lactic acid, and the like, into the milk. Lactic acid can be introduced into the milk by addition of a live culture of lactic acid-producing bacteria. Thus, the term “acidic agent” includes acids and acid-producing bacteria. In some embodiments, the pH is adjusted to below about 6.0; then curd formation is allowed to proceed. In other embodiments, the pH is adjusted to below about 6.0; rennet is added; and curd formation is allowed to proceed.

Cheeses that can be made using a method of the invention include any fresh, or ripened cheese that are made by a process that includes curd formation. Such cheese include, but are not limited to, Campesino, Chester, Danbo, Drabant, Herregard, Manchego, Provolone, Saint Paulin, Soft cheese, Taleggio, White cheese, Cheddar, Colby, Edam, Muenster, Gruyere, Emmenthal, Camembert, Parmesan, Romano, Mozzarella, Feta; cream cheese, Neufchatel, etc.

In some embodiments, the method further involves processing the cheese into a processed cheese food product. Processed cheese food products include, but are not limited to, pizza, ready-to-eat dishes, toast, burgers, lasagna, dressing, sauces, cheese powder, cheese flavor, and processed cheese.

Research Applications

The subject transgenic animals find use in research, to analyze the effects of increased levels of lysozyme on milk quality. In addition, the subject transgenic animals are useful for studying the regulation of transcription and translation of a lysozyme gene.

For example, mutations may be introduced into the lysozyme promoter region to determine the effect of altering expression in a lysozyme transgenic animal.

Lysozyme regulatory sequences incorporated into a transgene may be used to identify cis acting sequences required for transcriptional or translational regulation of expression, especially in different tissues or stages of development, and to identify cis acting sequences and trans-acting factors that regulate or mediate expression. Such transcription or translational control regions may be operably linked to a reporter gene or a lysozyme gene to examine the effects of the regulatory sequences on expression levels and mRNA stability.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric. Standard abbreviations are used, e.g., kb, kilobase(s); pl, picoliter(s); s, second(s); min, minute(s); hr, hour(s); and the like.

### Example 1

### Production of Lysozyme Transgenic Goats, and Characterization of Milk Produced From the Goats

Materials and Methods

Transgenic goats were generated by standard pronuclear microinjection with a DNA construct consisting of 23 kb of the promoter and 3′ regulatory elements of the bovine αₛ₁-casein gene coupled to the 540 bp cDNA for human lysozyme. Briefly, donor females (outbred dairy goats) were synchronized using progestin pessaries, superovulated with follicle stimulating hormone (FSH) and gonadotropin releasing hormone (GnRH), bred to fertile bucks and one-cell fertilized zygotes were collected from the oviducts. Zygotes with visible pronuclei were microinjected with approximately 2 pl of the DNA construct at a concentration of 5 ng/μl and surviving zygotes were then surgically transferred on the same day via midline laparotomy into the oviducts of synchronized recipient females. Pregnancies were carried to term and all offspring born were screened for the presence of the transgene with construct-specific polymerase chain reaction (PCR) primers. PCR-positive founders were confirmed by Southern blot. Expression of human lysozyme mRNA in the mammary gland was confirmed by standard northern blot analysis, with ³²P-labelled hLZ cDNA as probe, on total RNA isolated from sloughed mammary gland somatic cells collected from the milk. Positive founders were bred to non-transgenic males and F1 offspring were identified in a similar fashion.

Milk Collection

Milk samples were collected from individual transgenic and non-transgenic lactating females of the same parity. Milk was collected at parturition and then once a month for five months, corresponding to a total of six time-points at birth and months 1, 2, 3, 4, and 5. These time points include early and peak lactation. As the expression of human lysozyme in goat milk will be under the control of the bovine αₛ₁-casein gene, we expect the expression of lysozyme to follow a typical casein expression curve where levels start low and increase until peak lactation and then drop off. Lysozyme concentration in human milk also increases from colostrum to mature milk in humans (Sanchez-Pozo et al., 1986). Standard Dairy Herd Improvement Association (DHIA) analysis was conducted on fresh samples to determine the values for total solids, fat and protein content and somatic cell count was determined with a Fossimatic apparatus. Aliquots of milk were stored at 4° C. or −20° C. Fresh, refrigerated milk samples were used for the antimicrobial and functionality assays and frozen milk for western blot and activity analysis.

Western Analysis

Milk from all six time-points was analyzed for the presence, quantity and activity of human lysozyme. Standardized amounts of milk for all samples were subjected to standard 15% sodium dodecyl sulfate-polyacrylamide gel electrophoresis (SDS-PAGE) and transferred to a poly(vinylidene)fluoride PVDF membrane (Amersham) for western blotting with a 1:2500 dilution of a primary rabbit anit-human lysozyme polyclonal antibody (DAKO) followed by a 1:20000 dilution of a secondary goat anit-rabbit-horse radish peroxidase (HRP) antibody (BioRad). Signal was detected by enhanced chemiluminescence (ECL) (Amersham). Quantification of human lysozyme protein expression in goat milk was carried out by densitometry scanning of western blots with known amounts of human lysozyme standard as control. Milk samples were also tested for human lysozyme activity as previously described (Maga et al., 1995). Briefly, Micrococcus lysodeikticus (0.4%) was incorporated into a 15% SDS-PAGE gel, samples run, and the gel was placed in a renaturation buffer (50 mM sodium phosphate, 1% Triton-X100) at 37° C. for 1-2 days. Lysozyme activity is indicated by a clearing of the incorporated bacteria.

Antimicrobial Assays

The antimicrobial activity of human lysozyme transgenic goat milk and non-transgenic control milk was characterized with respect to the growth of key bacteria involved in mastitis, cold spoilage and cheese-making as previously described (Maga et al., 1998). Four classes of microorganisms chosen for use based on their significance to the dairy industry. The organisms include a nonpathogenic E. coliJ5, a cold spoilage-causing microorganism, Pseudomonas fragi (ATCC #4973), a lactic acid bacteria, Lactococcus lactis (ATCC #7962), and a clinical isolate of a mastitis-causing organism, Staphylococcus aureus, isolated and typed by the California Dairy Foods Safety Research Laboratory, University of California, Davis.

Four to five single colonies of bacteria were inoculated in 50 ml trypticase soy broth (TSB) and grown overnight at 37° C. One ml of the overnight culture was then inoculated in 50 ml of fresh medium and grown to log phase. The culture was centrifuged at 3500 rpm for 10 minutes at room temperature. The bacterial pellet was washed two times with 10 mM sodium phosphate buffer (pH 7.4) and resuspended in buffer. Ten μl of bacterial suspension adjusted to 1×10⁶ colony forming units (CFU)/ml was added to 50 μl of 1:10 diluted whole milk from either transgenic or non-transgenic goats. The final reaction volume was adjusted to 100 μl with buffer. The samples were incubated at 37° C. for up to 5 hours. To construct a growth curve, a 1:100 dilution at times 0, 30, 60, 90, 120, 180, 240 and 300 minutes after the start of incubation were plated on trypticase soy agar plates with a spiral palter. Plates were incubated 24 to 48 hours at 37° C. for the formation of distinct colonies and counted by machine. For these preliminary studies, all assays were conducted in duplicate and repeated two times. The differences between the mean number of colonies (CFU/ml) obtained for transgenic milk versus control milk were analyzed using a standard two-tailed, non-paired Student's t-test.

Processing Properties

The effects of in vivo-produced human lysozyme on several processing properties of milk were characterized. The rennet clotting time and curd strength of transgenic and non-transgenic control milk were evaluated as previously described (Maga et al. (1995) J. Dairy Sci.). The rennet clotting time of milk samples was determined by incubating 20 ml of 20° C. or 4° C. fresh milk with vegetable rennet (New England Cheesemaking Supply Company) at a final dilution of 1:4000 rennet:milk at 32° C. Samples were examined for formation of curd at 5-minute intervals. The rennet clotting time was taken as the time that had passed before a solid gel was formed at the bottom of the flask. Each experiment was done in duplicate and total of 8 experiments were carried out over two months. The mean rennet clotting time data was analyzed by a Student's t-test for statistical significance.

Curd strength was analyzed as reported (Maga et al., 1995). A controlled stress rheometer (AR1000 Rheometer; T. A. Instruments, New Castle, Del.; version 5.3 software) was used to evaluate the overall firmness of rennet-induced casein gels. A total of 20 ml of fresh, unrefrigerated 22° C. or refrigerated 4° C. whole milk samples were mixed with vegetable rennet (1:4000 vol/vol) at 20° C. and placed on the rheometer under a 4-cm diameter plate with a gap of 625 μm. The temperature was raised and held at 32° C. for 30 min for the formation of a gel and then lowered and held at 20° C. for 10 minutes before measurements are carried out (Renner-Nantz, 1994).

Gel formation will be detected by a continuous measurement of G* of the sample while heating at 32° C. Gelation will appear as a rapid increase of G* under the application of a small oscillatory stress at 1 Hz. After gelation and aging at 20° C. for 10 minutes, an oscillatory torque-sweep will be carried out over the range of 100 to 1000 dyne.cm at a frequency of 1 Hz. The values G*, a measure of the overall firmness of the gel, and tan delta, which is a measure of the degree of viscoelasticity and the type of bonds present (where tan delta is defined as the tangent of the phase shift, or the ratio of G″/G′, where G′ is a measure of the elasticity of the gel, and G″ is a measure of the viscosity of the gel) were measured. Barnes et al. (1989) “An Introduction to Rheology” Elsevier Publ., New York, N.Y. Two complete sets of measurements were taken for each gel formed. All experiments were repeated three times and the mean G* and tan delta values were analyzed by a Student's t-test for significance.

Results

To date we have identified one line of transgenic goats carrying and expressing the bovine αₛ₁-casein-human lysozyme transgene. The founder female of the transgenic line (goat 9135) also transmitted the transgene to her offspring. Of five F1 offspring produced to date, 3 have been transgenic, one female (goat 1022) and two males. Furthermore, all F1 goats also have passed the transgene to the F2 generation, producing one male and one female to date. These goats were identified as transgenic by PCR with two sets of primers specific for the transgene and confirmed by Southern blot. Expression of human lysozyme mRNA in the mammary gland was verified by northern blot analysis of total RNA purified from the somatic cells from the milk of lactating animals. Further results demonstrated that this line of goats expresses the human lysozyme protein in their milk and that the lysozyme is active. Human lysozyme protein expression in the milk was estimated to be 0.2 mg/ml as determined by western blotting. The percentage of milk yield that represents total fat and protein, as determined by testing through the California Dairy Herd Improvement Association testing program (DHIA) for the transgenic line fell in the same range as the averages of our dairy goat herd for both fat and protein. These goats now allow us to begin the preliminary characterization of the effect human lysozyme on milk.

Antimicrobial Effect

The effects of human lysozyme-containing transgenic goat milk on the growth of several milk-related strains of bacteria were determined. The lysozyme transgenic goat milk slowed the growth of nonpathogenic E. coli (Table 1). The mean number of colonies (CFU/ml) formed on plates incubated with lysozyme transgenic milk was significantly lower than on control plates with non-transgenic milk after 30, 60 and 90 minutes of incubation (P<0.05, P<0.001, P<0.001, respectively). The mean number of colonies on plates containing transgenic milk was also significantly lower than those on plates with ultra high temperature (UHT) pasteurized bovine milk at all time points (P<0.001 at 30 and 60 min, P<0.002 at 90 min). The nontransgenic control milk was different from UHT milk only after 30 minutes of incubation (P<0.01).

TABLE 1
Effect of human lysozyme transgenic milk on E. coli

Mean (CFU/ml ± SD) × 10⁵ after incubation (min)

| Goat | 30 | 60 | 90 |
|---|---|---|---|
| 9135* | 8.18 ± 2.49^{a,b} | 7.83 ± 2.34^{b,d} | 27.25 ± 16.77^{b,e} |
| | (n = 12) | (n = 12) | (n = 12) |
| Control** | 11.18 ± 2.72^{a,c} | 34.74 ± 10.58^{b} | 63.79 ± 11.49^{b} |
| | (n = 8) | (n = 8) | (n = 8) |
| UHT*** | 18.95 ± 6.07^{b,c} | 26.80 ± 5.47^{d} | 68.15 ± 28.30^{e} |
| | (n = 6) | (n = 6) | (n = 6) |

Means in the same column with the same subscript are significantly different.
^{a}P < 0.05,
^{b}P < 0.001,
^{c}P < 0.01,
^{d}P < 0.001,
^{e}P < 0.002
*Transgenic founder in second lactation expressing human lysozyme
**Non-transgenic control goat in second lactation
***Pasteurized bovine milk

Lysozyme transgenic milk slowed the growth of the cold-spoilage organism P. fragi (Table 2). Milk from the transgenic founder had fewer colonies than did milk from non-transgenic controls of the same lactation after both 120 and 180 minutes of incubation (P<0.01). Likewise, milk from the F1 transgenic goat resulted significantly less colonies being formed than did milk from controls of the same lactation at 120 and 180 minutes of incubation (P<0.02, P<0.1, respectively).

TABLE 2
Effect of human lysozyme transgenic milk on Pseudomonas fragi

| | Mean CFU/ml ± SD × 10⁵ after incubation (min) | | | | |
|---|---|---|---|---|---|
| Goat | 60 | 120 | 180 | 240 | 300 |
| 9135^{a} | 2.56 ± 0.23 | 0.77 ± 0.18* | 1.89 ± 0.13* | 0.82 ± 0.08 | 0.89 ± 0.27 |
| | (n = 2) | (n = 2) | (n = 2) | (n = 2) | (n = 2) |
| Control^{b} | 0.83 ± 0.51 | 1.30 ± 0.28 | 3.59 ± 1.06 | 1.65 ± 0.84 | 0.80 ± 0.39 |
| 2^{nd} lac | (n = 4) | (n = 4) | (n = 4) | (n = 4) | (n = 4) |
| 1022^{c} | 1.56 ± 0.31 | 0.94 ± 0.08** | 1.60 ± 0.48* | 0.53 ± 0.15 | 0.79 ± 0.04 |
| | (n = 2) | (n = 2) | (n = 2) | (n = 2) | (n = 2) |
| Control^{d} | 1.52 ± 0.66 | 2.10 ± 0.38 | 4.68 ± 1.84 | 2.29 ± 2.24 | 0.90 ± 0.26 |
| 1^{st} lac | (n = 4) | (n = 4) | (n = 4) | (n = 4) | (n = 4) |

^{a}Transgenic founder in second lactation expressing human lysozyme
^{b}Non-transgenic control goats (2 individuals) in second lactation
^{c}Transgenic F1 in first lactation expressing human lysozyme
HU dNon-transgenic control goats (2 individuals) in first lactation
*Transgenic goat was different from non-transgenic goats of the same lactation (P < 0.1)
**Transgenic goat was different from non-transgenic goats of the same lactation (P < 0.02)

Transgenic milk did not have significant effect on the growth of the mastitis-causing organism S. aureus, although despite the small sample size and great variation, results were trending towards significance (Table 3).

TABLE 3
Effect of human lysozyme transgenic milk on Staphylococcus aureus

| | Mean CFU/ml ± SD × 10⁵ after incubation (min) | | | | |
|---|---|---|---|---|---|
| Goat | 60 | 120 | 180 | 240 | 300 |
| 9135^{a} | 8.02 ± 3.23 | 7.67 ± 1.18 | 8.20 ± 6.58 | 12.7 + 2.83 | 9.23 ± 4.61 |
| | (n = 4) | (n = 4) | (n = 4) | (n = 2) | (n = 2) |
| Control^{b} | 9.09 ± 6.55 | 5.90 ± 1.39 | 5.75 ± 5.11 | 10.4 ± 2.07 | 12.4 ± 2.69 |
| 2^{nd} lac | (n = 4) | (n = 4) | (n = 4) | (n = 2) | (n = 2) |
| 1022^{c} | 13.7 ± 1.98 | 13.2 ± 6.03 | 11.0 ± 4.66 | 14.2 ± 5.73 | 35.6 ± 10.7 |
| | (n = 2) | (n = 2) | (n = 2) | (n = 2) | (n = 2) |
| Control^{d} | 8.07 ± 2.85 | 8.03 ± 2.73 | 17.4 ± 6.64 | 13.9 ± 9.79 | 41.8 ± 27.5 |
| 1^{st} lac | (n = 4) | (n = 4) | (n = 4) | (n = 4) | (n = 4) |

^{a}Transgenic founder in second lactation expressing human lysozyme
^{b}Non-transgenic control goats (1 individual) in second lactation
^{c}Transgenic F1 in first lactation expressing human lysozyme
^{d}Non-transgenic control goats (2 individuals) in first lactation

Lysozyme transgenic milk had no effect on the growth of the lactic acid bacteria, L. lactis (Table 4). In a preliminary study, levels of L. lactis grown in the presence of lysozyme-containing transgenic milk were not significantly different from levels of the bacteria grown with non-transgenic control milk.

TABLE 4
Effect of human lysozyme transgenic milk on Lactococcus lactis

| | Mean CFU/ml ± SD × 10⁵ after incubation (min) | | | | | |
|---|---|---|---|---|---|---|
| Goat | 0 | 60 | 120 | 180 | 240 | 300 |
| 9135^{a} | 2.33 ± 0.18 | 6.63 ± 5.19 | 8.54 ± 3.06 | 5.84 ± 3.50 | 6.63 ± 4.54 | 2.95 ± 0.58 |
| | (n = 2) | (n = 2) | (n = 2) | (n = 2) | (n = 2) | (n = 2) |
| Control^{b} | 13.8 ± 12.5 | 10.7 ± 8.78 | 7.41 ± 3.42 | 5.47 ± 3.99 | 9.50 ± 7.48 | 6.18 ± 9.12 |
| 2^{nd} lac | (n = 2) | (n = 2) | (n = 2) | (n = 2) | (n = 2) | (n = 2) |
| 1022^{c} | 7.40 ± 3.12 | 13.2 ± 2.76 | 17.9 ± 1.62 | 4.70 ± 1.61 | 1.94 ± 0.11 | TNTC |
| | (n = 2) | (n = 2) | (n = 2) | (n = 2) | (n = 2) | |
| Control^{d} | 6.85 ± 3.73 | 8.44 ± 6.70 | 9.95 ± 3.58 | 6.10 ± 2.32 | 3.79 ± 3.78 | 10.6 ± 7.68 |
| 1^{st} lac | (n = 4) | (n = 2) | (n = 2) | (n = 2) | (n = 2) | (n = 2) |
| Transgenic | 4.87 ± 3.45 | 9.89 ± 5.07 | 13.2 ± 5.74 | 5.27 ± 2.32 | 4.29 ± 3.77 | 2.95 ± 0.58 |
| | (n = 4) | (n = 4) | (n = 4) | (n = 4) | (n = 4) | (n = 2) |
| Control | 10.3 ± 9.29 | 9.58 ± 7.33 | 8.68 ± 3.51 | 5.78 ± 3.04 | 6.64 ± 6.28 | 8.40 ± 8.15 |
| | (n = 8) | (n = 8) | (n = 8) | (n = 8) | (n = 8) | (n = 8) |
| UHT | ND | 8.71 ± 8.47 | 20.2 ± 14.6 | 18.2 ± 22.2 | 0.81 ± 0.77 | TNTC |
| | | (n = 2) | (n = 2) | (n = 2) | (n = 2) | |

^{a}Transgenic founder in second lactation expressing human lysozyme
^{b}Non-transgenic control goats (1 individual) in second lactation
^{c}Transgenic F1 in first lactation expressing human lysozyme
^{d}Non-transgenic control goats (2 individuals) in first lactation
TNTC—Too numerous to count
ND—Not determined

Effect on Processing Properties

Human lysozyme transgenic milk was also capable of affecting several of the processing properties of milk. The rennet clotting time of rennet-induced gels was significantly faster (6 minutes, P<0.03) when lysozyme transgenic milk was used compared to nontransgenic controls (Table 5). The rennet clotting time was not different between samples used fresh at 22° C. or stored at 4° C. The gel strength of rennet-induced gels, as measured by G*, was significantly stronger when transgenic goat milk was used compared to nontransgenic controls of the same lactation (Table 5). The tanδ value, or a measure of the types of bonds present, in transgenic milk was not different from the nontransgenic controls (Table 5).

TABLE 5
Physical properties of goat milk expressing human lysozyme

| Goat | RCT¹ (min) | G* ²(Pa) | tanδ³ |
|---|---|---|---|
| 9135^{a} | 17 ± 7.7* | 28 ± 13.2** | 0.33 ± 0.16 |
| | (n = 19) | (n = 3, df = 58) | (n = 3, df = 58) |
| Control^{b} | 23 ± 12.7 | 21 ± 13.8 | 0.30 ± 0.62 |
| | (n = 41) | (n = 3, df = 138) | (n = 3, df = 138) |

^{a}Transgenic founder in second lactation expressing human lysozyme
^{b}Non-transgenic control goats (4 individuals) in second lactation
¹Rennet clotting time
²Overall gel strength (Pa);
G* values over the torque range of 1 to 10 Pa.
³Degree of viscoelasticity (types of bonds present);
tanδ values over the torque range of 1 to 10 Pa.
*Transgenic goat was different from non-transgenic goats of the same lactation (P < 0.03).
**Transgenic goat was different from non-transgenic goats of the same lactation (P < 0.001).

Health and Bacterial Make-Up of the Udder

A measure of udder health was made by monitoring the somatic cell count (SCC) of milk taken from both halves of the udder. In lactating dairy herds, milk production, quality and SCC are routinely monitored by California Dairy Herd Improvement Association (DHIA) analysis. A measure of the number of somatic cells per ml of milk provides an indication of the prevalence of mastitis in an animal. The SSC over a period of two years were significantly lower (P<0.02) in human lysozyme (HLZ) transgenic animals compared with our control herd over the same period (Table 6), indicating improved udder health in the HLZ transgenic animals. The in vivo growth of bacteria in the udder and those introduced into the milk during the process of milking was evaluated in both HLZ transgenic and non-transgenic control animals.

TABLE 6
Production parameters of HLZ transgenic and control goat milk

| | % Fat | | % Protein | | SCC | |
|---|---|---|---|---|---|---|
| Parity | Transgenic | Herd | Transgenic | Herd | Transgenic | Herd |
| 1^{st} | 3.11 | 3.24 | 2.90 | 2.67 | 272,000 | 436,000 |
| 2^{nd} | 2.96 | 2.95 | 2.67 | 2.56 | 269,000 | 506,000 |
| 3^{rd} | 3.45 | 3.32 | 2.76 | 2.46 | 319,000 | 523,000 |
| All | 3.17 | 3.22 | 2.78 | 2.51 | 275,000* | 533,000 |

Milk production indicators including the weight percent fat, weight percent protein and somatic cell count (SCC) were determined by DHIA analysis for transgenic goats in their first, second and third lactation and compared to non-transgenic control goats raised in the same herd. Values for transgenic animals significantly different from non-transgenic controls (herd) according to Student's t-test are indicated with one asterisk (P<0.02, df=20).

It is evident from the above Examples that the invention provides lysozyme transgenic ungulates that produce in their milk a higher level of lysozyme than a non-transgenic ungulate of the same species, and that the milk produced by a subject transgenic ungulate has a significantly reduced rennet clotting time, and significantly greater bacteriostatic activity, than milk from a non-transgenic ungulate of the same species. In addition, cheese made from the milk of a subject transgenic animal has increased gel strength compared to cheese made from the milk of a non-transgenic ungulate of the same species.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A transgenic ungulate comprising a transgene encoding lysozyme.

2. The transgenic ungulate of claim 1, wherein said ungulate is female, and wherein said lysozyme-encoding transgene is expressed in mammary gland cells of said ungulate, and wherein milk produced by said transgenic ungulate has a level of lysozyme that is at least about 10% higher than the level of lysozyme in milk of a non-transgenic ungulate of the same species.

3. The transgenic ungulate of claim 2, wherein said transgene comprises a coding sequence for lysozyme operably linked to a mammary specific promoter.

4. The transgenic ungulate of claim 1, wherein said ungulate is male.

5. The transgenic ungulate of claim 1, wherein said ungulate is a goat.

6. The transgenic ungulate of claim 1, wherein said transgene is chromosomally integrated.

7. The transgenic ungulate of claim 1, wherein said ungulate is heterozygous for the transgene.

8. The transgenic ungulate of claim 1, wherein said ungulate is homozygous for the transgene.

9. The transgenic ungulate of claim 1, wherein the transgene encodes human lysozyme.

10. An isolated fertilized egg, wherein said egg is isolated from a transgenic ungulate comprising a transgene encoding lysozyme.

11. A composition comprising an isolated fertilized egg according to claim 10; and a cryoprotective agent.

12. A method of producing a food product, said method comprising harvesting a food product from a transgenic ungulate of claim 2.

13. A method of producing a food product, the method comprising processing a food product harvested from a transgenic ungulate of claim 2.

14. A method of producing cheese, the method comprising:
a) adding rennet to milk harvested from a transgenic ungulate according to claim 2;
b) allowing curd formation to occur; and
c) producing cheese from the curds.

15. A milk product produced by a transgenic ungulate of claim 2, wherein said milk product has a level of lysozyme that is at least about 10% higher than the level of lysozyme in milk of a non-transgenic ungulate of the same species.

16. The milk product of claim 15, wherein the milk product has reduced rennet clotting time compared to a milk product of a non-transgenic animal of the same species.

17. A processed milk produced from milk of a transgenic ungulate of claim 2.

18. The processed milk product of claim 17, wherein the processed milk product is cheese.

19. The processed milk product of claim 18, wherein the cheese has increased gel strength compared to cheese made from milk of a non-transgenic ungulate of the same species.
